(19) United States
(12) Patent Application Publication
(10) Pub. No.: US 2022/0047686 A1
(43) Pub. Date: Feb. 17, 2022

(54) METHODS FOR TREATING INFECTIOUS DISEASES

(71) Applicant: Mayo Foundation for Medical Education and Research, Rochester, MN (US)

(72) Inventor: Samuel J. Asirvatham, Rochester, MN (US)

(21) Appl. No.: 17/384,039

(22) Filed: Jul. 23, 2021

Related U.S. Application Data
(60) Provisional application No. 63/066,015, filed on Aug. 14, 2020.

(51) Int. Cl. A61K 38/48; A61K 9/00; A61K 41/00; A61K 31/4706; A61K 31/4709; A61K 31/4965; A61K 31/529; A61N 1/32; A61M 15/00

(52) U.S. Cl. CPC A61K 38/4813; A61K 9/0073; A61K 41/0047; A61K 31/4706; A61K 31/4709; A61M 2205/054; A61K 31/529; A61N 1/327; A61M 15/009; A61M 2202/064; A61K 31/4965

(57) ABSTRACT

Various methods and devices of treating infectious diseases are described. In one example embodiment, a method of treating an infectious disease in an individual in need thereof is provided, including administering a therapeutic agent to the individual and electroporating a tissue of the individual, wherein the therapeutic agent is inhalable.

### CROSS-REFERENCE TO RELATED APPLICATIONS

This application claims the benefit of U.S. Provisional Application Ser. No. 63/066,015, filed Aug. 14, 2020. The disclosure of the prior application is considered part of (and is incorporated by reference in) the disclosure of this application.

### BACKGROUND

### 1. Technical Field

This document relates to methods for treating infectious diseases. For example, this document relates to methods including inhalation-based therapies and/or electroporation as a treatment for infectious diseases.

### 2. Background Information

Respiratory infectious diseases, such as coronavirus disease 2019 (COVID-19) caused by the novel severe acute respiratory syndrome coronavirus 2 (SARS-CoV-2), remains a major worldwide health problem. To date, SARS-CoV-2 has caused over 11 million confirmed infections globally, leading to over 500,000 deaths, and making it a public health emergency of international concern. Nonetheless, no specific antiviral drug or vaccine for COVID-19 treatment exists yet. The high infectivity and the increasing fatality of COVID-19 highlight the demand for the discovery of novel treatments.

Electroporation is a technique that uses a high voltage, rapid burst of current to non-thermally introduce multiple nano-pores within the cells' walls of surrounding tissue, specifically within the lipid bilayer of the cell membranes as a result of the electrical field. Depending on the voltage and frequency used, these pores can be reversible (i.e., increase the permeability of these cell to chemotherapeutic agents) and/or irreversible (i.e., trigger cell death by the process of apoptosis rather than necrosis). Given the different composition of each cell-type membrane, along with other discrepancies, electroporation can allow for a differential effect on different tissues.

A unique challenge with potential therapeutic agents for the treatment of infectious diseases, such as COVID-19, is the delivery of an effective dose to the tissues of interest, which can often be hindered by factors such as pre-systemic and systemic clearance. Thus, there is a need for effective drug delivery of therapeutic agents and/or alternative drug delivery routes for the treatment of infectious diseases.

### SUMMARY

This document describes methods for the treatment of infectious diseases. For example, this document describes methods and materials for the delivery of inhalation-based therapies and/or electroporation as a treatment for infectious diseases.

In one aspect, this disclosure is directed to a method of treating an infectious disease. The method can include administering a therapeutic agent to the individual, and electroporating a tissue of the individual to enhance the absorption or uptake of the therapeutic agent, wherein the therapeutic agent is inhalable.

In some embodiments, the infectious disease comprises at least one of a viral infectious disease or a bacterial infectious disease. In certain aspects, the infectious disease comprises at least one of a coronavirus disease 2019 (COVID-19), Middle East Respiratory Syndrome (MERS), severe acute respiratory syndrome (SARS), viral pneumonia, bacterial pneumonia, common cold, pharyngitis, bronchitis, broncholitis, acute laryngotracheobronchitis, tracheitis, tracheobronchitis, sinusitis, or laryngotracheitis. In some embodiments, the infectious disease includes at least one of a prion disease or an antimicrobial resistant disease. In certain aspects, the infectious disease is coronavirus disease 2019 (COVID-19). In some embodiments, the therapeutic agent is an ion channel blocker. In certain aspects, ion channel blocker is a voltage-gated ion channel blocker. In some embodiments, the voltage-gated ion channel blocker comprises at least one of chloroquine, hydroxychloroquine, or quinidine.

In certain aspects, the ion channel blocker is a non-voltage-gated ion channel blocker. In some embodiments, the non-voltage-gated ion channel blocker comprises at least one of amiloride or tetrodotoxin. In certain aspects, the therapeutic agent is an antibody that neutralizes a human coronavirus. In some embodiments, the human coronavirus comprises at least one of 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), or SARS-CoV2. In certain aspects, the therapeutic agent is a recombinant human angiotensin converting enzyme 2 (rhACE2). In some embodiments, the therapeutic agent is aerosolized or nebulized.

In certain aspects, administering the therapeutic agent includes administering the therapeutic agent via an inhalation drug delivery device. In some embodiments, the drug delivery device is a dry powder inhaler, a nebulizer, or a pressurized metered-dose inhaler. In certain aspects, the tissue is a respiratory tissue. In some embodiments, the respiratory tissue comprises at least one of a nasal cavity, trachea, bronchi, bronchiole, lung, alveolus, pulmonary blood vessel, pharynx, larynx, sinus, pulmonary pleura, or respiratory cilia.

In certain aspects, the step of electroporating the tissue can include positioning a first electrode intra-bronchially in the individual, positioning a second electrode intrabronchially in the individual, and delivering an electric stimulation to the tissue via the first and second electrodes. In some embodiments, the step of electroporating the tissue includes delivering a sodium chloride solution to the individual, positioning an electrode in contact with the surface of a chest of the individual, and delivering an electric stimulation to the tissue via the sodium chloride solution and the electrode. In certain aspects, the therapeutic agent comprises sodium chloride. In some embodiments, the step of electroporating the tissue includes positioning a first electrode within a first lumen of a first blood vessel of the individual, positioning a second electrode within a second lumen of a second blood vessel of the individual, and delivering an electric stimulation to the tissue via the first and second electrodes. In certain aspects, the blood vessel is a pulmonary artery, pulmonary vein, inferior vena cava, or superior vena cava.

In another aspect, this disclosure is directed to a method of treating an infectious disease in an individual in need thereof The method can include electroporating a respiratory tissue of the individual, wherein the infectious disease comprises at least one of a viral infectious disease or a bacterial infectious disease.

In another aspect, this disclosure is directed to a method of treating an infectious disease in an individual in need thereof The method can include administering an inhalable therapeutic agent to the individual, wherein the inhalable therapeutic agent comprises at least one of an ion channel blocker, an antibody that neutralizes a human coronavirus, or a recombinant human angiotensin converting enzyme 2 (rhACE2).

In another aspect, this disclosure is directed to a method of vaccinating an individual in need thereof. The method can include administering an inhalable recombinant human angiotensin converting enzyme 2 (rhACE2) bound to a human coronavirus.

In yet another aspect, this disclosure is directed to a method of treating an infectious disease in an individual in need thereof The method can include administering a topical recombinant human angiotensin converting enzyme 2 (rhACE2).

In yet another aspect, this disclosure is directed to a method of treating a disorder of the renin-angiotensin-aldosterone system in an individual in need thereof. The method can include administering a therapeutic agent to the individual, wherein the inhalable therapeutic agent comprises at least one of an ion channel blocker, an antibody that neutralizes a human coronavirus, or a recombinant human angiotensin converting enzyme 2 (rhACE2).

In some embodiments, the therapeutic agent is administered via at least one of an oral route, a topical route, an inhaled route, or a parenteral route. In certain aspects, the method can further include electroporating a tissue of the individual.

Particular embodiments of the subject matter described in this document can be implemented to realize one or more of the following advantages. The methods described herein can improve the bioavailability of a therapeutic agent for the treatment of an infectious disease. For example, when administered in combination, the localized delivery of electroporation and an inhalation-based therapeutic agent can improve the bioavailability of the therapeutic agent in the tissues presenting the most significant pathogenic (e.g., viral) loads (e.g., respiratory tissues). In some embodiments, another advantage of the methods described herein includes delivery a therapeutic agent directly to the respiratory tract, thereby bypassing systemic clearance of the body. In another aspect, an additional advantage of the methods described herein is the ability to use external or internal physical methods (e.g., pulsed direct current fields) to prevent viral pathogenicity. Furthermore, in some embodiments, the methods described herein have improved safety and efficacy than conventional treatments. Due to the delivery of therapeutic agents locally, systemic side effects associated with current and/or potential drug candidates can be avoided. In some embodiments, electroporation may be a standalone antiviral, bacterial, fungal, or other pathogenic infection process treatment. In some embodiments, the potential for increasing intra-cellular concentration of a drug or therapeutic agent locally (e.g., within a pathogen in the body and/or within a cell invaded by the pathogen) where the infection is present may allow combination therapy with drugs and/or therapeutic agents at low doses (e.g., as compared to standard-of-care doses and/or doses required to achieve efficacy), which otherwise would be ineffective.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the invention are set forth in the accompanying drawings and the description below. Other features, objects, and advantages of the invention will be apparent from the description, drawings, and from the claims.

### DETAILED DESCRIPTION

This document describes methods and materials for the treatment of infectious diseases. For example, this document describes methods and devices for delivering electroporation and/or inhalation-based therapeutic agents.

Serious infectious diseases (e.g., viral pneumonias) include the necessity for pathogens (e.g., viruses) to interact with host receptors in the respiratory tract and are characterized by the intracellular adoption of the pathogen.

The methods disclosed herein provide physical methods to prevent viral pathogenicity including the use of pulsed direct current fields (e.g., electroporation). Electroporation is a technique that uses a high voltage rapid burst of current to non-thermally introduce multiple nano-pores within the cells' walls of surrounding tissue, specifically within the lipid bilayer of the cell membranes as a result of the electrical field. The high voltage rapid burst of current can be delivered to cells infected with pathogens, including viruses, and can consequently, reduce or eliminate their pathogenicity.

Furthermore, the methods described herein can provide increased safety and efficacy of therapeutic agents by administering the agents via alternative drug delivery routes, such as, but not limited to an inhalation route or a topical route, which specifically target tissues having a high pathogen load. These alternative drug delivery routes can also help combat the transmission infectious diseases (e.g., COVID-19) and reduce the risk of exposure by decreasing pathogenicity.

In an aspect, the present disclosure is directed to methods of treating infectious diseases in an individual in need thereof. The method can include administering a therapeutic agent to the individual, and electroporating a tissue of the individual. The therapeutic agent can be an inhalable therapeutic agent.

### Infectious Diseases

In some embodiments, the infectious disease includes a viral infectious disease, a bacterial infectious disease, or both. For example, the infectious disease can be caused by viral pathogens such as, but not limited to 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), or SARS-CoV2. In some embodiments, the infectious disease includes any viral or other infectious pathogen that uses or generates a biofilm. In some embodiments, the infectious disease includes any viral or other infectious pathogen that makes chemotherapeutic options difficult (e.g., antimicrobial resistant infectious diseases). could be targeted with the electroporation approach. In some embodiments, the infectious disease includes viral carrier states, chronic bacterial infections such as mycobacteria, prion diseases, fungal disorders, or any combination thereof. In other examples, the infectious disease can be caused by bacterial pathogens such as gram-positive and/or gram-negative bacteria such as, but not limited to Streptococcus pneumoniae, Klebsiella pneumoniae, Pseudomonas aeruginosa, and Staphylococcus aureus. In some embodiments, the infectious disease includes prion diseases, any viral, bacterial, or fungal diseases that colonize to create a carrier state, uses a biofilm to shield against traditional chemotherapeutic and antibiotic-type agents, and/or infects and colonizes within tumors and mechanical devices (e.g., stents, valves, leads, prosthetic devices, etc.).

In some embodiments, viral infectious diseases include a coronavirus disease 2019 (COVID-19), Middle East Respiratory Syndrome (MERS), severe acute respiratory syndrome (SARS), viral pneumonia, bacterial pneumonia, common cold, pharyngitis, bronchitis, broncholitis, acute laryngotracheobronchitis, tracheitis, tracheobronchitis, sinusitis, laryngotracheitis, or any combination thereof. In some embodiments, the infectious disease is coronavirus disease 2019 (COVID-19). In some embodiments, the infectious disease is a disorder caused by a SARS-CoV-2 infection.

### Therapeutic Agents

The spike protein (S-protein) of coronaviruses interacts with cell receptors to mediate viral entry into target cells. Additional evidence has suggested that both SARS-CoV and SARS-CoV-2 employ angiotensin-converting enzyme 2 (ACE2) as the entry receptor and that the receptor-binding domain (RBD) of the S-protein directly binds to ACE2, triggering endocytosis of virus particles. ACE2 is not only a functional receptor of coronaviruses, but also acts as an important negative regulator of the renin-angiotensin system (RAS) through conversion of the vasoconstrictor angiotensin II (Ang II) to its metabolite angiotensin-(1-7) (Ang 1-7) and angiotensin I (Ang I) to angiotensin-(1-9) (Ang 1-9). The ACE2/Ang 1-7 axis plays a series of roles in the improvement of endothelial dysfunction, anti-inflammation, anti-hypertension, anti-thrombus, and anti-fibrosis activity, and cardiovascular protection. The protective effect of ACE2 is associated with attenuating Ang II levels and increasing Ang 1-7 levels in lung pathophysiology. Thus, RAS signaling and ACE2 can play crucial roles in SARS-CoV- and SARS-CoV-2-induced disorders.

Ion channels are pore-forming protein complexes that facilitate the flow of ions across the hydrophobic core of cell membranes. They are present in the plasma membrane and membranes of intracellular organelles of all cells, performing essential physiological functions including establishing and shaping the electrical signals which underlie muscle contraction/relaxation and neuronal signal transmission, neurotransmitter release, cognition, hormone secretion, sensory transduction and maintaining electrolyte balance and blood pressure. They are usually classified by gating i.e., the stimulus that “opens” the channel, be it chemical or mechanical stimuli.

Ion channel blockers are molecules that are used to prevent the opening of ion channels in order to produce a physiological response in a cell. Ion channel blockers can affect the receptivity of the ACE2 receptor. Ion channel blockers can, therefore, block or reduce the binding affinity or the number of binding events of a pathogen (e.g., SARS-CoV-2) to an ACE2 receptor. In some embodiments, the therapeutic agent is an ion channel blocker. The ion channel blocker can be a voltage-gated ion channel blocker. The voltage-gated ion channel blocker can include at least one of chloroquine, hydroxychloroquine, or quinidine. Systemic delivery of voltage-gated ion channel blockers is known to produce side effects (e.g., cardiac ion channel toxicity), which are a major issue. In some embodiments, the voltage-gated ion channel blockers are aerosolized. Using the respiratory route for direct, local delivery of the voltage-gated ion channel blockers can allow achievement of therapeutic efficacy without organ toxicity.

In some embodiments, the ion channel blocker is a non-voltage-gated ion channel blocker. For example, non-voltage-gated ion channels can include ligand-gated ion channels. In some embodiments, the non-voltage-gated ion channel blocker is a non-voltage-gated sodium channel blocker. In some embodiments, the non-voltage-gated ion channel blocker includes at least one of amiloride or tetrodotoxin.

Non-voltage-gated sodium channels are not typically found in the heart or other important conducting or neural tissue; therefore, therapeutic efficacy with minimal risk can be achieved.

In other examples, the therapeutic agent is an antibody that neutralizes a human coronavirus. For example, the therapeutic agent can reduce a viral load of a pathogen to less than about 0-50% of the viral load prior to administering the therapeutic agent. In some embodiments, the neutralizing antibody prevents viral adherence with its receptor (e.g., ACE2). In some examples, the human coronavirus that is neutralized includes at least one of 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), or SARS-CoV2.

In some embodiments, the neutralizing antibody includes inhaled or systemically injected neutralizing antibodies possibly combined to prevent attachment of viruses to their receptor sites in the epithelium.

In some aspects, the methods disclosed herein include blocking, reducing, or preventing antigen receptor binding via administration of a recombinant receptor molecule itself (e.g., ACE2); thus, the recombinant receptor molecule can bind with free viruses (e.g., SARS-CoV or SARS-CoV-2) prior to entering the interface with the host tissue.

In some embodiments, the therapeutic agent is a recombinant human angiotensin converting enzyme 2 (rhACE2). The rhACE2 can bind to free SARS-CoV or SARS-CoV-2, thereby reducing or eliminating the number of viral particles that can bind to endogenous ACE in an individual. In some embodiments, rhACE2 blocks SARS-CoV or SARS-CoV-2 from infecting an individual. In some embodiments, the therapeutic agent is a fusion protein including one or more portions of rhACE2. In some examples, the therapeutic agent is aerosolized or nebulized. In some embodiments, the therapeutic ageing is a topical recombinant human angiotensin converting enzyme 2 (rhACE2). In some embodiments, the topical rhACE2 is delivered to the skin, oral mucosa, or both. In some embodiments, the topical rhACE2 is applied as a coating to wearable devices (e.g., protective masks) to further increase efficacy in preventing viral entrance.

In another embodiment, the rhACE2 is aerosolized and mixed with aerosolized viral particles (e.g., SARS-CoV or SARS-CoV-2) to create an inhalable vaccine. In some embodiments, the inhalable vaccine can be therapeutically administered to an individual in need thereof to treat the pathogenic disease (e.g., SARS-CoV-2 infection). In some embodiments, the inhalable vaccine can be prophylactically administered to an individual in need thereof to stimulate development of immunity against the pathogenic disease (e.g., SARS-CoV-2 infection).

### Delivery of Therapeutic Agents

The methods disclosed herein include administering the therapeutic agent to the individual in need thereof alone or in combination with electroporation of a tissue of the individual. In some embodiments, the therapeutic device is an inhalable therapeutic agent. The inhalable therapeutic agent can be delivered via an inhalation drug delivery device. The drug delivery device can be a dry powder inhaler, a nebulizer, or a pressurized metered-dose inhaler. In other examples, the therapeutic agent can be delivered topically.

In some embodiments, the methods of the disclosure include electroporating a tissue of the individual. In some embodiments, the tissue is a tissue with a high pathogenic load. In some embodiments, the tissue is a respiratory tissue. For example, the respiratory tissue can include at least one of a nasal cavity, trachea, bronchi, bronchiole, lung, alveolus, pulmonary blood vessel, pharynx, larynx, sinus, pulmonary pleura, or respiratory cilia.

The step of electroporating the tissue can include, for example, positioning an electrode intra-bronchially in the individual, and delivering an electric stimulation to the tissue via the electrode. The electrode can be placed intra-bronchially using, for example, a bronchoscope.

The method can include electroporating the tissue by positioning an electrode in contact with the surface of a chest of the individual and delivering an electric stimulation to the tissue via the electrode. In this example, the therapeutic agent includes sodium chloride that can act as a virtual electrode. In other embodiments, the method can include electroporating the tissue by positioning an electrode within the lumen of a blood vessel of the individual and delivering an electric stimulation to the tissue via the electrode. In some examples the blood vessel can include a pulmonary artery, pulmonary vein, inferior vena cava, or superior vena cava.

In some examples, the method can include electroporating the tissue by positioning one or more delivery electrodes (e.g., anodes or cathodes) in a first bronchus (e.g., either in the main stem or a secondary or tertiary order bronchus), positioning one or more return electrodes (e.g., anodes or cathodes) in a second bronchus (e.g., either in the main stem or a secondary or tertiary order bronchus) either ipsilateral or contralaterally, and delivering an electric stimulation to the tissue via the one or more anodes and one or more return electrodes.

In some examples, the method can include electroporating the tissue by positioning one or more electrodes (e.g., anodes or cathodes) in a first branch of the pulmonary artery, positioning one or more electrodes (e.g., anodes or cathodes) in a second branch of the pulmonary artery of the ipsilateral or contralateral node or in a neighboring pulmonary vein, and delivering an electric stimulation to the tissue via the one or more anodes and one or more return electrodes.

In some examples, in order to facilitate more effective bronchial drug delivery using electroporation, electrodes for electroporation can be placed (e.g., via a bronchoscope) into the bronchial tree. In addition, surface electrodes and electrodes in the pulmonary arterial circulation can be used in combination with the electrodes in the bronchial tree. Such an arrangement of electrodes can create advantageous vectors to help the inhalational agent penetrate through the mucosa of the bronchus.

In some examples, the method can include electroporating the tissue by delivering a sodium chloride solution to the patient, positioning one or more floating electrodes (e.g., anodes or cathodes) intrabronchially, positioning one or more surface patches on the skin of the chest of the patient, and delivering an electric stimulation to the tissue via the one or more anodes and one or more return electrodes. The one or more floating electrodes may serve as a delivery electrode and the one or more surface patches on the skin of the chest may serve as a return electrode. In some embodiments, the sodium chloride solution may be in the form of liquid droplets, an aerosol, a spray, a mixture of gas and solid particles, a mixture of gas and liquid particles, or any combination thereof.

In some examples, once the electrodes are positioned, a pulsed DC current can be delivered for varying intervals (e.g., few seconds to several minutes) and for several, varying frequencies until a desired response either to the neighboring cells or the infectious agent is noted. In some embodiments, the DC current may provide electroporating energy. The electroporation delivery sequence may be delivered intermittently with quick testing for pathogen presence (e.g., via polymerase chain reaction (PCR) assays or other microbiological techniques) done between electroporation deliveries to assess for response.

In some embodiments, the energy may be delivered using one or more return electrodes that may be positioned intra-bronchially and/or extra-bronchially. Extra-bronchial return electrodes may be located internally within the patient, e.g., within the pleural space, etc. and/or on the skin of the patient. Some potentially useful extra-bronchial placements for return electrodes may include, e.g., in the mediastinum; individualized positioning on the skin of the patient (e.g., placing the electrode(s) behind the right lower lobe); in the azygous vein; in the superior vena cava or other vascular structures; or peribronchial placement (e.g., either via exiting the bronchus and/or through mediastinoscopy. In some embodiments, the return electrode may also be a virtual electrode in the form of saline or other fluid provided in, e.g., the peri-bronchial and/or extra-bronchial spaces.

In some embodiments, inhalational agents plus electroporation can be used as a method to permanently treat bronchial asthma. For example, in some embodiments an agent can be delivered in combination with electroporation to impair the function of smooth bronchial muscle tissue lining. This inventive concept uses differential vectors (i.e., delivering electroporation from the surface rather than within the bronchial lumen) to deliver one or more inhalational agents (i.e., to drive a therapeutic agent such as botulinum toxin, anti-smooth muscle agents and drugs, or bronchodilators) into the bronchial smooth muscle. The therapeutic agent would paralyze bronchial smooth muscle tissue temporarily or permanently as a way of treating asthma. Said another way, this technique includes the delivery of anti-infective, paralytic, and modulatory agents in an inhaled manner in combination with differential vector electroporation. In some embodiments, an electroporation electrode (or electrodes) located intra-bronchially could function as the return electrode while energy is delivered through one or more electrodes positioned peri-bronchially and/or extra-bronchially. Although one of the reasons for the extra-bronchial and/or pen-bronchial placement of an electrode is for smooth muscle ablation from a potentially superior vantage point, in some embodiments, extra-bronchial and/or pen-bronchial placement of electrodes and the delivery of energy using those electrodes may provide for specific manipulation, modulation and ablation of the autonomic nervous system relevant to the bronchial smooth muscle to which the energy is delivered as a part of the electroporation process in combination with the inhalation of one or more inhalational agents to treat asthma.

In another aspect, the present disclosure is directed to methods of treating a disorder of the renin-angiotensin-aldosterone system in an individual in need thereof. The method can include administering a therapeutic agent to the individual. The inhalable therapeutic agent can include at least one of an ion channel blocker, an antibody that neutralizes a human coronavirus, or a recombinant human angiotensin converting enzyme 2 (rhACE2). The therapeutic agent can be administered via at least one of an oral route, a topical route, an inhaled route, or a parenteral route. In some embodiments, the method of treating a disorder of the renin-angiotensin-aldosterone system can be administered alone or in combination with electroporation a tissue of the individual. In some embodiments, examples of disorders of the renin-angiotensin-aldosterone system include hypertension, cardiac rhythm disturbances, disorders of mineralocorticoid secretion, prevention of lung toxicity from agents such as amiodarone, or any combination thereof.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the process depicted in the accompanying figures does not necessarily require the particular order shown, or sequential order, to achieve desirable results. In certain implementations, multitasking and parallel processing may be advantageous.

What is claimed is:

1. A method of treating a disease in an individual in need thereof, the method comprising:
administering, via inhalation, a therapeutic agent to the individual; and
while administering the therapeutic agent, electroporating a tissue of the individual to enhance uptake or absorption of the therapeutic agent into the tissue.

2. The method of claim 1, wherein the disease comprises at least one of a viral infectious disease or a bacterial infectious disease.

3. The method of claim 1, wherein the disease comprises at least one of: a coronavirus disease 2019 (COVID-19), Middle East Respiratory Syndrome (MERS), severe acute respiratory syndrome (SARS), viral pneumonia, bacterial pneumonia, common cold, pharyngitis, bronchitis, broncholitis, acute laryngotracheobronchitis, tracheitis, tracheobronchitis, sinusitis, and laryngotracheitis.

4. The method of claim 1, wherein the disease comprises at least one of a prion disease or an antimicrobial resistant disease.

5. The method of claim 1, wherein the disease is coronavirus disease 2019 (COVID-19).

6. The method of claim 1, wherein the therapeutic agent is an ion channel blocker.

7. The method of claim 6, wherein the ion channel blocker is a voltage-gated ion channel blocker.

8. The method of claim 7, wherein the voltage-gated ion channel blocker comprises at least one of chloroquine, hydroxychloroquine, or quinidine.

9. The method of claim 6, wherein the ion channel blocker is a non-voltage-gated ion channel blocker.

10. The method of claim 9, wherein the non-voltage-gated ion channel blocker comprises at least one of amiloride or tetrodotoxin.

11. The method of claim 1, wherein the therapeutic agent is an antibody that neutralizes a human coronavirus.

12. The method of claim 11, wherein the human coronavirus comprises at least one of 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), or SARS-CoV2.

13. The method of claim 1, wherein the therapeutic agent is a recombinant human angiotensin converting enzyme 2 (rhACE2).

14. The method of claim 1, wherein the therapeutic agent is aerosolized or nebulized.

15. The method of claim 1, wherein administering the therapeutic agent comprises administering the therapeutic agent via an inhalation drug delivery device.

16. The method of claim 15, wherein the drug delivery device is a dry powder inhaler, a nebulizer, or a pressurized metered-dose inhaler.

17. The method of claim 1, wherein the tissue comprises at least one of: a nasal cavity, trachea, bronchi, bronchiole, lung, alveolus, pulmonary blood vessel, pharynx, larynx, sinus, pulmonary pleura, and respiratory cilia.

18. The method of claim 1, wherein the step of electroporating the tissue comprises:
positioning a first electrode intra-bronchially in the individual;
positioning a second electrode intrabronchially in the individual; and
delivering an electric stimulation to the tissue via the first and second electrodes.

19. The method of claim 1, wherein the step of electroporating the tissue comprises:
delivering a sodium chloride solution to the individual;
positioning an electrode in contact with the surface of a chest of the individual; and
delivering an electric stimulation to the tissue via the sodium chloride solution and the electrode.

20. The method of claim 1, wherein the step of electroporating the tissue comprises:
positioning a first electrode within a lumen of a pulmonary artery, pulmonary vein, inferior vena cava, or superior vena cava of the individual;
positioning a second electrode within a lumen of a second blood vessel of the individual; and
delivering an electric stimulation to the tissue via the first and second electrodes.
